(19) United States
(12) Patent Application Publication
(10) Pub. No.: US 2026/0026767 A1
(43) Pub. Date: Jan. 29, 2026

(54) METHOD FOR DETERMINING AN ESTIMATION OF A SCATTERED RADIATION DISTRIBUTION AND FOR TRAINING AT LEAST ONE TRAINED MODEL, PROCESSING DEVICE, X-RAY DEVICE, COMPUTER PROGRAM, AND DATA MEDIUM

(71) Applicant: Siemens Healthineers AG, Forchheim (DE)

(72) Inventor: Philipp Roser, Erlangen (DE)

(21) Appl. No.: 19/279,231

(22) Filed: Jul. 24, 2025

(30) Foreign Application Priority Data
Jul. 24, 2024 (DE) 10 2024 206 982.5

(51) Int. Cl. A61B 6/00

(52) U.S. Cl. CPC A61B 6/5294; A61B 6/483

(57) ABSTRACT

A computer-implemented method for determining an estimation of a two-dimensional scattered radiation distribution in a two-dimensional X-ray image that is based on an imaging procedure performed by an X-ray detector is provided. The method includes receiving the X-ray image, applying an estimation algorithm to the X-ray image or to a two-dimensional intermediate image determined from the X-ray image. The estimation algorithm determines a respective two-dimensional partial scattered radiation image for a plurality of physical scatter processes such that image values of pixels of the respective partial scattered radiation image in each case describe an estimated value for a respective scattered radiation dose that was applied to a respective detector region of the X-ray detector assigned to the respective pixel during acquisition of the X-ray image by the respective physical scatter process. The estimation of the scattered radiation distribution is determined based on the plurality of partial scattered radiation images.

This application claims the benefit of German Patent Application No. DE 10 2024 206 982.5, filed on Jul. 24, 2024, which is hereby incorporated by reference in its entirety.

### BACKGROUND

The present embodiments relate to determining an estimation of a two-dimensional scattered radiation distribution in a two-dimensional X-ray image that is based on an imaging procedure using an X-ray detector.

Scattered radiation has a significant impact on the image quality that is achievable in the course of an X-ray imaging procedure. In the field of computed tomography, for example, the presence of scattered radiation may lead to streak artifacts, unsharpness, or a low-frequency distortion of the image contrast.

To reduce such scattered radiation artifacts, an anti-scatter grid may be arranged in front of the X-ray detector. However, parts of the primary radiation are also intercepted by anti-scatter grids that, at a given X-ray dose, may lead to lower image quality, or as a result of which, higher X-ray doses may be required.

In certain fields of application (e.g., in neurovascular imaging for the purpose of detecting aneurysms or an embolic stroke by imaging of the brain), it is therefore typical already today to do without the use of antiscatter grids. In this case, scattered radiation artifacts may be reduced by increasing the distance between the patient and the detector. In addition or alternatively, a software-based scattered radiation correction may be used.

A software-based scattered radiation correction may in principle be achieved by simulation of the X-ray imaging (e.g., by solving the Boltzmann transport equation or by a Monte Carlo method). However, due to the high computational overhead required for a correction of the type, such corrections are poorly suited when an imaging procedure is to be performed with low latency (e.g., at least approximately in real time, such as in order to accompany a medical intervention).

The same problems also occur with what are termed empirical methods, in which a scattered radiation effect is realized by optimizing the projection images based on an image quality measure for reconstructed slices. With this approach, it is namely necessary to repeat the image reconstruction multiple times, which is likewise very computationally intensive.

For a scattered radiation correction involving comparatively low computational overhead, approaches are therefore increasingly adopted that use a model trained by machine learning. The model immediately determines an image corrected for scattered radiation from a projection image. In this case, however, good results in which the effect of scattered radiation is markedly reduced and distortions of the X-ray image due to the processing are avoided may be achieved only when the model has been trained for a specific embodiment of the imaging (e.g., for a specific imaging geometry, specific imaging parameters, and/or a specific patient group). A generalization is not easily possible since with this approach the image processing is strongly influenced by the image datasets used for the training.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved approach to scattered radiation estimation or scattered radiation correction that is suitable for interventional imaging or generally for low-latency imaging and, for example, may also be used in a flexible manner for a variety of imaging tasks is provided.

In one embodiment, a computer-implemented method for determining an estimation of a two-dimensional scattered radiation distribution in a two-dimensional X-ray image is provided. The method is based on an imaging procedure using an X-ray detector and includes the following acts: receiving the X-ray image; applying an estimation algorithm to the X-ray image or to a two-dimensional intermediate image determined from the X-ray image, where the estimation algorithm determines a respective two-dimensional partial scattered radiation image for a plurality of physical scatter processes such that image values of pixels of the respective partial scattered radiation image in each case describe an estimated value for a respective scattered radiation dose that was applied to a respective detector region of the X-ray detector assigned to the respective pixel during acquisition of the X-ray image by the respective physical scatter process; and determining the estimation of the scattered radiation distribution based on the plurality of partial scattered radiation images.

By the separate determination of the partial scattered radiation images for the different physical scatter processes, the proposed correction may also be explained by the underlying physics of the X-ray and takes account of the physics when the estimation algorithm is not based directly on the underlying physics in the sense of a simulation or similar but, for example, also when the estimation algorithm has been implemented at least in part by an algorithm trained using machine learning or by a plurality of such algorithms. Suitable approaches to the training of the estimation algorithm or its subalgorithms will be explained in greater detail later.

Taking different scattered radiation images into account for different physical scatter processes, for example, enables separate subalgorithms to be used for determining the partial scattered radiation images, thus allowing less complex algorithms to be used. This may also simplify a training since typically, given lower complexity of the algorithm to be trained, fewer free parameters of the algorithm that need to be determined in the training are present, which provides that less training data is required for the training.

Further, as will be explained in more detail later, by using the different partial scattered radiation images or, as the case may be, by evaluating the different physical scatter processes, additional consistency checks may also be performed in the course of the determination of the partial scattered radiation images or the training of the estimation algorithm or its subalgorithms. The robustness of the scattered radiation estimation may be further improved as a result.

For example, an X-ray attenuation image that describes an attenuation of X-ray radiation radiated through an object (e.g., through a patient) that is arranged between an X-ray source and a detector region of the X-ray detector assigned to the respective pixel of the X-ray attenuation image may be determined as an intermediate image. For example, the attenuation may be described on a logarithmic scale, as is standard practice in the field of X-ray imaging (e.g., medical X-ray imaging).

The attenuation may be calculated based on the original X-ray intensity or X-ray dose I₀ and the X-ray intensity or X-ray dose I incident in the respective detector region during the imaging (e.g., as aₜₒₜ=−log (I/I₀)).

An X-ray image, in the context of the present description, is understood, for example, as an image that describes the X-ray intensity or dose received in the detector region that is assigned to the respective pixel. However, it is also possible, for example, that the X-ray image already describes a relative X-ray intensity or dose (e.g., the ratio I/I₀), or that the respective attenuation aₜₒₜ is described directly by the X-ray image.

The estimation algorithm or at least a subalgorithm of the estimation algorithm may be or include a model trained by machine learning. In general, a model trained by machine learning mimics the cognitive functions that bring human beings into contact with thought processes of other human beings. By a training based on training data, the trained model is able, for example, to adapt to new circumstances and to recognize and extrapolate patterns. Another term for “model trained by means of machine learning” is “trained function.”

Although trained models may learn complex relationships, the use of more complex trained models also is frequently possible at least approximately in real time. The at least one trained model may therefore learn in the course of the training, for example, to provide results that substantially correspond to the results of a more complex calculation method used during the training, for example by a solving of the Boltzmann transport equation or of a Monte Carlo method. Thus, by using a trained model, a similarly good estimation may be achieved as may be achieved based on significantly more complex calculations, such that, in spite of good estimation quality, for example, a quasi-real-time capability of the scattered radiation estimation and, for example, also a correction of the X-ray image based hereon may be achieved. Accordingly, for example, the quality of a scattered radiation correction during an interventional imaging procedure may be substantially improved by the described approach.

Compared with the immediate correction of an X-ray image by a trained model as explained in the introduction, however, the estimation algorithm nonetheless remains physically motivated by the determination of separate partial scattered radiation images for different physical scatter processes. As a result of this, the intermediate results of the estimation algorithm may be drawn upon in the course of the training and/or immediately during the application of the estimation algorithm in order, for example, to identify and discard physically unrealistic results or to suppress the generation of such results in the course of the training.

Generally, the parameters of a machine learning model may be adjusted by training in order to provide the trained model. The training may be conducted, for example, in advance of the method according to the present embodiments and consequently not be part of the method according to the present embodiments. Alternatively, it would, however, also be possible to conduct the training in the form of additional upstream method steps within the method according to the present embodiments.

For example, supervised training, semi-supervised training, unsupervised training, reinforcement learning, and/or active learning may be used. In addition, representation learning, which is also referred to as “feature learning,” may also be used. The parameters of the machine learning models may be adjusted, for example, iteratively using multiple training steps. For example, a determined cost function may be minimized in the course of the training. For example, the backpropagation algorithm may be used in the training, of a neural network, for example.

A machine learning model may, for example, include a neural network, a support vector machine, a decision tree, and/or a Bayesian network, and/or a transformer, and/or the machine learning model may be based on k-means clustering, Q-learning, genetic algorithms, and/or association rules. For example, a neural network may be a deep neural network, a convolutional neural network, or a convolutional deep neural network. Further, a neural network may be an adversarial network, a deep adversarial network, and/or a generative adversarial network.

The model trained by machine learning may be based on supervised training using training datasets, each of which includes source data and a plurality of desired results. The source data serves directly or after preprocessing as input data for the estimation algorithm, where the respective desired result is to be provided as the respective partial scattered radiation image by the estimation algorithm during a processing of the respective input data. At least the desired results are based on a simulation of the X-ray imaging.

The simulation of the X-ray imaging may, as explained above in relation to the training of the model, be carried out, for example, in advance of the method according to the present embodiments and consequently not be part of the method according to the present embodiments. Alternatively, however, such a simulation may form an additional upstream method step of the method according to the present embodiments.

The X-ray imaging may be simulated, for example, by a solving of the Boltzmann transport equation or by a Monte Carlo method or a raycasting. The simulation may, for example, use known parameters of a simulated X-ray imaging device and a three-dimensional model of an object or a patient to be imaged in the course of the simulation. The three-dimensional model may describe, for example, a spatial distribution of different materials or of scatter and interaction characteristics of the imaged material. In the simplest case, such a three-dimensional model may be generated synthetically (e.g., based on an anatomical atlas and known scatter and interaction characteristics of different body parts), or such a three-dimensional model may also be generated manually.

For example, the source data of the respective training dataset (e.g., an X-ray image or an X-ray attenuation image) may be generated in the course of such a simulation.

However, it may also be advantageous for at least some of the training datasets or also for all the training datasets to draw upon X-ray images that are based on an actual X-ray imaging procedure as source data or as a basis of the source data. In this case, the three-dimensional model of the imaged object or patient may be generated based on the image data of the same X-ray imaging procedure in order, as explained above, to determine the partial scattered radiation images assigned to the respective X-ray image as desired results by simulation of the X-ray imaging.

For example, the X-ray image of the respective training dataset may be acquired as part of a computed tomography scan, whereby the image data of the X-ray imaging describes a three-dimensional representation of the imaged object or patient. By a classification of different image areas (e.g., using an algorithm trained by machine learning or by use of an anatomical atlas to which the three-dimensional representation is registered), the different areas of the three-dimensional representation may then be classified, for example, as tissue, bone, etc., whereby the at least approximately known interaction and scatter characteristics of the respective imaged material may be associated with them. For the sake of completeness, it is pointed out that in principle the interaction and scatter characteristics of a three-dimensional object may also be estimated from individual X-ray images. For example, additional information about a known imaging geometry and the object may be referred to.

Using the estimation algorithm, separate partial scattered radiation images for at least two or for all the physical scatter processes may be determined from the group including Rayleigh scattering, Compton scattering, and multiple scattering. It has been recognized that the scattered radiation actually incident on the X-ray detector may be estimated with a good degree of accuracy by taking into account a plurality or, for example, all of the cited scatter processes.

The estimation algorithm may include a first subalgorithm that processes the X-ray image or the intermediate image as input data and determines as output data a respective partial attenuation image for a plurality of physical interaction processes of the X-ray radiation with irradiated material. Image values of the respective partial attenuation image in each case describe an estimation of the local attenuation of the X-ray radiation by the respective physical interaction processes. A second subalgorithm of the estimation algorithm determines the partial scattered radiation images as a function of the partial attenuation images.

For example, the first subalgorithm and/or the second subalgorithm may in each case be a model trained by machine learning or includes at least one such model. As will be explained in more detail later, for example, an end-to-end training of the entire estimation algorithm may be carried out.

Since the different interaction processes contribute to different degrees and in different ways to the ultimately measured scattered radiation, it is beneficial to determine the partial attenuation images initially as an intermediate step in the estimation algorithm. Because the first subalgorithm therefore decomposes the total X-ray attenuation into different partial attenuations due to different interaction processes, the first subalgorithm may also be referred to as a decomposition algorithm or, in the event of being trained by machine learning, as a trained decomposition model.

As already mentioned above, in an X-ray imaging procedure (e.g., in the field of medical imaging), it is entirely in line with standard practice to image, not the registered intensities, but an attenuation, so that, for example, bones appear brighter than surrounding tissue. In the described method, use is made of the fact that the attenuation of X-ray radiation may typically be described completely at least approximately by a few absorption and scatter processes.

On account of the training, a trained model, similarly to a human observer, may distinguish already based on the X-ray image or, as explained above, from the X-ray attenuation image determined from the X-ray image, between different irradiated materials (e.g., between bone and tissue). Since the portions of the different interaction processes in the attenuation of the X-ray radiation are dependent primarily on the irradiated material, the respective portion of the attenuation due to the different interaction processes, and consequently the individual partial attenuation images, may be determined with a good degree of accuracy already based on a single X-ray image or X-ray attenuation image. Some of the considered interaction processes may correspond to a respective process of the considered physical scatter processes. In one embodiment, however, at least one interaction process that corresponds to none of the considered physical scatter processes is taken into account.

In one embodiment, partial attenuation images for at least two or all of the physical interaction processes may be determined from the group including Rayleigh scattering, Compton scattering, and photoelectric absorption. Thus, for example, as well as the Rayleigh and Compton scatterings already taken into consideration as physical scatter processes, the photoelectric absorption may additionally be taken into account in which no immediate photon emission occurs, but which may nonetheless contribute significantly to the attenuation in an X-ray imaging procedure.

Since the attenuation of the X-ray radiation during a multiple scattering is dominated by the first scatter or absorption process, the possibility of a multiple scattering does not explicitly need to be taken into account with regard to the attenuation, but may be taken into account indirectly via the other interaction processes.

The second subalgorithm may, for example, include a number of partial algorithms, where a respective one of the partial algorithms: determines a partial scattered radiation image for a Rayleigh scattering as the physical scatter process and processes the partial attenuation images for the Rayleigh scattering and the Compton scattering; and/or determines a partial scattered radiation image for a Compton scattering as the physical scatter process and processes the partial attenuation images for the Compton scattering and the photoelectric absorption; and/or determines a partial scattered radiation image for a multiple scattering as the physical scatter process and processes the partial attenuation images for the Compton scattering, the Rayleigh scattering, and the photoelectric absorption.

By separating the second subalgorithm into multiple partial algorithms, there results a lesser complexity for the individual partial algorithm than would be required for a monolithic second subalgorithm. This may reduce the computational overhead for the evaluation of the second subalgorithm or at least enable the evaluation to be completed faster (e.g., by a possible parallelization of the partial algorithms). Further, in an implementation of the respective partial algorithm as a model trained by machine learning, a high degree of robustness may be achieved already with a relatively small number of training datasets as a result of such a reduction in complexity. This holds true, for example, since it has been recognized in the course of the development of the present embodiments that in order to determine an estimation of at least some of the partial scattered radiation images, as described above, only parts of the partial attenuation images are to be taken into account as input data in order to achieve a robust scattered radiation estimation.

For example, all the partial algorithms may additionally process the X-ray image or supplementary input data determined from the X-ray image. For example a quotient from the original X-ray intensity or X-ray dose I₀ and the X-ray intensity incident in the respective detector region assigned to the pixel during the imaging or X-ray dose I may be used as the supplementary input data.

The first subalgorithm and/or the second subalgorithm may in each case be a model trained by machine learning or include at least one model trained by machine learning. The respective trained model is based on a supervised training using training datasets, each of which includes source data and a plurality of desired results. The source data serves directly or after a preprocessing as input data for the estimation algorithm and hence for the first subalgorithm. The respective desired result is to be provided as the respective partial scattered radiation image in a processing of the respective input data by the estimation algorithm and therefore by the second subalgorithm.

It is therefore proposed to use an estimation algorithm that has been trained by an end-to-end training, where, as will be explained later, intermediate results (e.g., the partial attenuation images) may, however, be taken into account in addition in the course of the training. Training data for such an end-to-end training may be provided, for example, by a simulation of the imaging.

Although an end-to-end training is performed, a subdivision of the estimation algorithm into subalgorithms is typically advantageous. The complexity of the individual subalgorithm is reduced as a result, which provides that the training overhead may also be reduced. The subdivision of the estimation algorithm into subalgorithms, by taking account of the partial attenuation images in the course of the training, first enables the physics underlying the imaging to be explicitly taken into consideration and consequently to provide that a physically motivated behavior of the estimation algorithm is realized, and/or second, to validate a physical plausibility of the estimation using the partial attenuation images in the course of the application of the trained model. For example, a scattered radiation estimation may be discarded or supplemented by a corresponding alert if the sum of the partial attenuation images deviates all too strongly from the processed X-ray attenuation image. In the event of such a deviation, it is possible, for example, to revert to a fallback algorithm for scattered radiation estimation or correction, which, for example, may use a less accurate approach for the scattered radiation estimation.

An end-to-end-training may be implemented, for example, such that in the course of the training, a cost function is minimized or generally optimized, which is dependent on a measure for the deviation of those partial scattered radiation images determined for the respective training input dataset by the estimation algorithm from respective target partial scattered radiation images specified by the desired result of the respective training dataset. The optimization of the cost function may be implemented using per se known optimization approaches (e.g., using a gradient descent method).

The training may include an optimization of a cost function, where the cost function is dependent on image values of the pixels of the partial attenuation images and/or where the optimization is performed subject to a side condition evaluating the image values of the partial attenuation images. For example, the cost function may be dependent on the above-explained measure for the deviation of the partial scattered radiation images from the target partial scattered radiation images, and in addition (e.g., as a further summand of a weighted sum forming the cost function), on the image values of the pixels of the partial attenuation images.

The side condition may, for example, require that the sum of the attenuations specified by the partial attenuation images for the respective pixel corresponds to the total attenuation for the respective pixels specified by an X-ray attenuation image (e.g., by the intermediate image).

The side condition may be taken into account, for example, by using the known method of Lagrange multipliers or the additional term resulting in this case due to the side conditions may be included in the cost function (e.g., as a summand of a weighted sum). Accordingly, if a differentiable model is used, as is common practice in the field of machine learning, there still results a differentiable optimization problem even when the side condition is taken into account, whereby, for example, a gradient descent method may continue to be used in the course of a backpropagation in order to perform a training of the model, or of the plurality of models used in the estimation algorithm, using machine learning.

The fulfillment of the side condition and/or the cost function may be dependent on an attenuation deviation for the respective training dataset that specifies a measure for the deviation of the image values of the pixels of an X-ray attenuation image specified by the respective source data or determined from the source data from a respective comparative value. The comparative value corresponds to the sum of the image values of all of the partial attenuation images for the respective pixel.

For example, the side conditions or one of a plurality of side conditions may require that the attenuation deviations are zero for all pixels and/or the attenuation deviations may be taken into account in the cost function such that an increase in the respective attenuation deviation leads to a less optimal cost function (e.g., increases the value of a cost function that is to be minimized).

In addition or alternatively, the fulfillment of the side condition and/or the cost function may be dependent on at least one partial attenuation estimation determined for the respective training dataset. The at least one partial attenuation estimation is based on a classification of the matter irradiated by the X-ray radiation exclusively based on the X-ray image or X-ray attenuation image specified by the source data of the respective training dataset. The respective partial attenuation estimation describes an estimated attenuation of the X-ray radiation by a respective process of the interaction processes in the respective pixel of the X-ray image or X-ray attenuation image.

The cost function may be dependent, for example, on a deviation of the respective partial attenuation estimation from the respective partial attenuation image such that such a deviation is minimized, for example, in the course of the optimization of the cost function in compliance with the other optimization criteria. The partial attenuation estimation may be based, for example, on a classification into a few classes (e.g., into just 2 or 3 classes). For example, a distinction may be made exclusively between pixels that image irradiated bone and pixels that image only tissue, which may be modeled approximately as water. Such a classification may be determined, for example, by consideration of the local first and/or second derivatives of a lowpass-filtered X-ray attenuation image provided as an intermediate image or determined from the X-ray image since, for example, bones surrounded by tissue generate an attenuation profile with initially rising and subsequently falling attenuation along a line in the image. By consideration of the spatial derivative of the attenuation, it is therefore possible to robustly identify bone-imaging regions of the X-ray image or X-ray attenuation image. Since the interaction of water and bone with X-ray radiation is known per se, a partial attenuation estimation may be made on this basis.

Compared with a simulation or similar, for example, a partial attenuation estimation determined as explained above is relatively inaccurate. For this reason, by taking it into account in the training, the accuracy of the estimation of the scattered radiation distribution when the trained model is used in cases in which the processing X-ray image is very similar to the X-ray images present in the training datasets used, may potentially drop. However, since the partial attenuation estimation is based on very general principles, this results in the training being generalized up to a certain degree beyond the training data used such that an overfitting of the model in the course of the training may be avoided. Taking the partial attenuation estimation into account for at least one interaction process in the course of the training therefore acts as a regularization of the training. If a weighted sum is optimized as a cost function, a regularization term dependent on the partial attenuation estimations may be weighted more strongly or more weakly in order to adjust the degree of regularization.

In addition or alternatively, the fulfillment of the side condition and/or the cost function may be dependent on the relative magnitudes of image values of the same pixel in different images of the partial attenuation images. Based on the properties of the material typically irradiated in the course of the medical imaging, for example, it may be assumed, for example, that the interaction due to Rayleigh scattering leads both to a lower attenuation than the interaction due to Compton scattering and to a lower attenuation than the interaction due to photoelectric absorption. For this reason, at least for a majority of the pixels or, for example, on average over all of the pixels, the image value of the partial attenuation image for the Rayleigh scattering should also be less than the respective image value of the respective partial attenuation image for the Compton scattering and for the photoelectric absorption. The image values of the partial attenuation images for the Compton scattering and for the photoelectric absorption may be relatively similar such that, for example, their difference is less than the respective difference from the image value of the partial attenuation image for the Rayleigh scattering.

Based on the cited relationships, the fulfillment of the side condition and/or the cost function may be dependent, for example, on quotients or differences of image values of the same pixel for different partial attenuation images and/or on quotients or differences of the average values of all of the image values of different partial attenuation images.

In addition or alternatively, the respective training dataset may additionally include a target image for at least one of the partial attenuation images. The cost function may then be dependent in addition on a measure for the deviation of the respective target image from the associated partial attenuation image. The target images may be determined, for example, in the course of that simulation of the imaging that is also used in order to determine the desired results for the respective training dataset. By additionally taking into account the target images for the partial attenuation images, a supervised training of the first subalgorithm may be combined with the above-explained end-to-end training of the entire estimation algorithm.

The present embodiments further relate to a computer-implemented method for training at least one trained model, by which the estimation algorithm or at least a respective subalgorithm or partial algorithm of the estimation algorithm is implemented in the computer-implemented method for determining an estimation of a two-dimensional scattered radiation distribution using machine learning. The computer-implemented method includes the steps: receiving training datasets, each of which includes source data and a plurality of desired results, where the source data is in each case a two-dimensional X-ray image or a two-dimensional intermediate image that is based on an X-ray imaging procedure or a simulation of an X-ray imaging procedure, and where the respective desired result specifies a respective partial scattered radiation image that is to be provided by the estimation algorithm during a processing of the source data of the respective training dataset; training the at least one trained model using a supervised learning based on the training datasets; and providing the at least one trained model.

The two-dimensional X-ray image may be based, for example, on an X-ray imaging procedure or a simulation of an X-ray imaging procedure. The intermediate image may be determined from such an X-ray image or specified directly by a simulation. As already explained above, the intermediate image may relate, for example, to a total attenuation of an X-ray radiation radiated onto the object or the patient by the object or patient in the respective pixel.

Further features that are explained in the context of the computer-implemented method according to the present embodiments for determining an estimation of a two-dimensional scattered radiation distribution may also be applied with the advantages cited there to the computer-implemented method for training at least one trained model, and vice versa.

The present embodiments also relate to a processing device that is configured for performing the computer-implemented method. The processing device may be embodied, for example, as suitably programmed data processing devices, or alternatively, the cited functionality may be implemented at least in part as hardwired. The processing device may be integrated into a medical imaging apparatus (e.g., into an X-ray machine) or be embodied separately from the latter. The processing device may be implemented, for example, as a workstation computer, server, or cloud solution.

The present embodiments additionally relate to an X-ray device including an X-ray source and an imaging X-ray detector that includes a processing device according to the present embodiments. As a result of integrating a processing device according to the present embodiments, and consequently as a result of implementing the method according to the present embodiments in an X-ray device, the explained scattered radiation estimation or a scattered radiation correction based hereon may be performed directly in the course of the data acquisition or during a visualization for a user using the X-ray device.

The present embodiments also relate to a computer program including instructions that are configured to perform the computer-implemented method according to the present embodiments when the computer program is executed on a data processing device.

The present embodiments also relate to a data medium (e.g., a non-transitory computer-readable storage medium) including the computer program according to the present embodiments.

Features that are discussed in the context of one of the methods according to the present embodiments or one of the devices according to the present embodiments may also be applied together with the cited advantages to the other disclosed subject matters of the present embodiments.

### BRIEF DESCRIPTION OF THE DRAWINGS

Further advantages and specific details of the invention will become apparent from the following exemplary embodiments as well as from the associated schematic drawings, in which:

FIG. 1 shows an example embodiment of an X-ray device that includes an example embodiment of a processing device;

FIG. 2 shows a flowchart of an example embodiment of a computer-implemented method for determining an estimation of a two-dimensional scattered radiation distribution in a two-dimensional X-ray image;

FIG. 3 shows a flowchart of an example embodiment of the computer-implemented method for training at least one trained model used in FIG. 2 using machine learning; and

FIG. 4 shows an example structure of the trained model.

### DETAILED DESCRIPTION

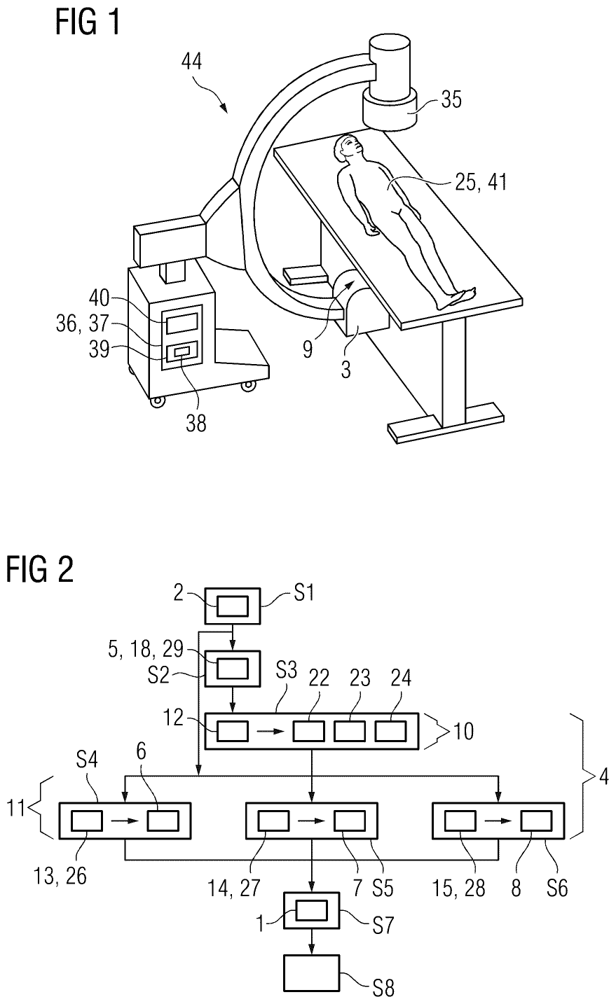

FIG. 1 shows an X-ray device 44 that is configured for acquiring two-dimensional image data of a patient 41. The patient 41 is irradiated with X-ray radiation by an X-ray source 35. The X-ray radiation attenuated by matter 25 of the patient 41 impinges on an X-ray detector 3 that includes a plurality of detector regions 9. The X-ray intensity or X-ray dose incident in the respective detector region 9 is measured in order to specify a respective image value of a respective pixel of the X-ray image 2.

However, since the interaction of the X-ray radiation with the matter 25 of the patient 41 is not limited to a pure absorption of the X-ray radiation, but scatter processes also occur, the X-ray detector 3 is also impinged on by scattered radiation, which may interfere with the imaging. In addition or alternatively to reducing the detected scattered radiation by other approaches (e.g., using antiscatter grids), it is therefore beneficial to perform an estimation of a scattered radiation distribution in the X-ray image (e.g., in order to correct the X-ray image subsequently as a function of the determined estimation, such as by subtraction of the estimation or by scaling or iterative multiplication of the detected X-ray intensities as a function of the estimation).

For this reason, a method for determining an estimation 1 of a two-dimensional scattered radiation distribution in a two-dimensional X-ray image 2 is implemented by the processing device 36, which in the example, is integrated in the X-ray device 44, though in principle may also be embodied separately therefrom (e.g., as a workstation computer, server, or cloud solution). An example embodiment of such a method will be explained in the following with reference to FIG. 2.

In the example, the method is implemented by a computer program 38 that is stored in the memory of a data processing device 37 and is executed by its freely programmable processor 40. The data processing device 37 programmed in this way therefore implements the processing device 36. The processor may be, for example, a microprocessor, a microcontroller, an SOC, or also an FPGA. However, the functionality may also be implemented partly or completely as hardwired (e.g., as an ASIC).

In the method illustrated as a flowchart in FIG. 2, the estimation 1 of the scattered radiation distribution is determined such that initially an estimation algorithm 4 is applied to a two-dimensional intermediate image 5 determined from the X-ray image 1 in order to determine a respective two-dimensional partial scattered radiation image 6-8 for a plurality of physical scatter processes. In this case, the image values of pixels of the respective partial scattered radiation image 6-8 each specify an estimated value for a respective scattered radiation dose that was radiated onto the respective detector region 9 of the X-ray detector 3 assigned to the respective pixel during acquisition of the X-ray image 1 by the respective physical scatter process. The estimation 1 of the scattered radiation distribution is then determined based on the plurality of partial scattered radiation images 6-8 (e.g., by summation of the partial scattered radiation images 6-8).

In practice, the X-ray image 2 is first received in act S1. In the example shown in FIG. 1, the X-ray image 1 may be provided by the acquisition of the X-ray image. The X-ray image may therefore be received by a hardware component performing the acquisition or, for example, also by another software component likewise implemented by the data processing device 37. In principle, however, it is also possible, for example, to receive an X-ray image already acquired earlier (e.g., from a server).

In act S2, the X-ray image 2, which in the example describes respective X-ray doses received in the respective detector region 9 in the course of the imaging, is converted into an intermediate image 5. In the example, an X-ray attenuation image is generated in the process by logarithmizing a quotient from the respective image value of the X-ray image and a known applied X-ray dose and a subsequent change of sign. The X-ray attenuation image forms the input data 18 for the estimation algorithm 4 unrelated in the example in practice for a first subalgorithm 10 of the estimation algorithm 4.

In act S3, the intermediate image 5 is processed by the first subalgorithm 10 or, in the example, using a model 12 trained by machine learning and implementing the first subalgorithm 10 in order to provide a plurality of partial attenuation images 22-24 as output data. In this case, the image values of the respective partial attenuation image 22-24 each describe an estimation of the local attenuation of the X-ray radiation by a respective process of the physical interaction processes. In this context, a Rayleigh scattering, a Compton scattering, and a photoelectric absorption are taken into account in the example as physical interaction processes.

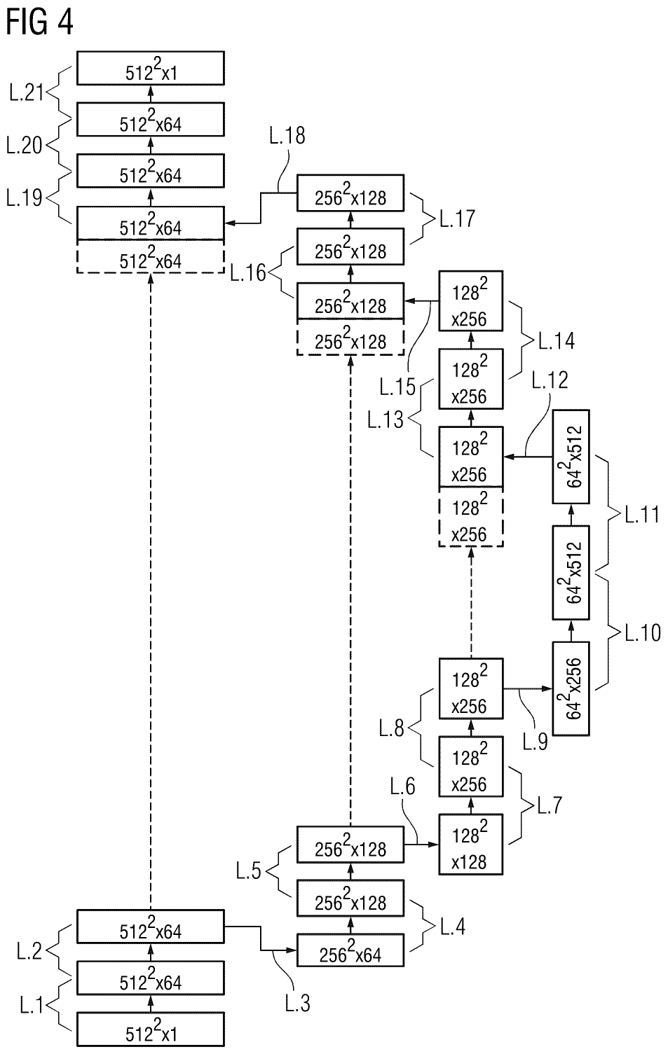

The trained model 12 may in this case be, for example, a convolutional neural network that may be configured, for example, as a U-Net. An example of a suitable U-Net will be explained in more detail later with reference to FIG. 4. In this case, in the example shown there, it is assumed for the sake of simplicity that image data having only one image layer (e.g., precisely one image value per pixel) is used both as input data 18 and as output data of the trained model 12. In order to provide multiple partial attenuation images, however, a plurality of layers may be used, at least on the output side, thereby increasing the number of nodes in the network proportionately. Alternatively, it would be possible, for example, to use three separate U-Nets as the trained model 12, of which each generates one of the partial attenuation images as output data.

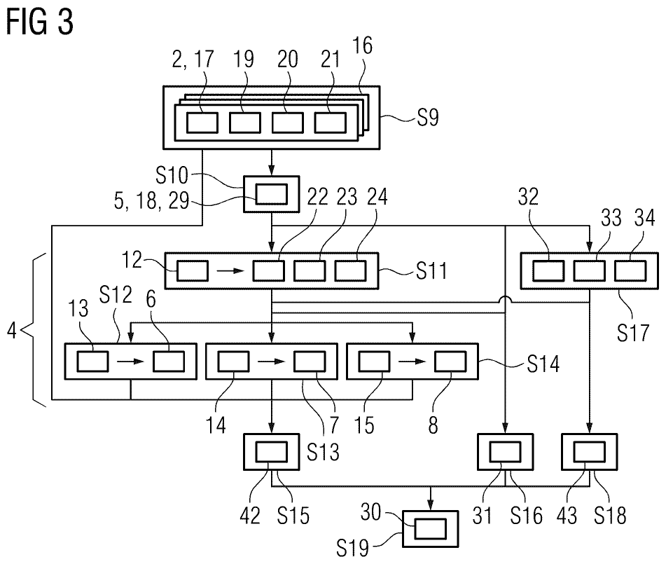

A suitable example of a possible method of training the trained model 12 will be explained later with reference to FIG. 3.

Acts S4-S6 implement a second subalgorithm 11 of the estimation algorithm 4 that determines the partial scattered radiation images 6-8 as a function of the partial attenuation images 22-24. In the example, the second subalgorithm 11 is implemented by three partial algorithms 26-28 executed in parallel, each of which generates one of the partial scattered radiation images 6-8.

In the example, the partial algorithms 26-28 are also implemented by a respective model 13 15 trained by machine learning. The trained models 13-15 also may be formed in each case by a convolutional neural network, which may have a U-Net structure, for example. Since, in the example, as is explained in more detail below, a plurality of partial attenuation images 20-24 are processed in each case as input data by the respective trained model 13-15, a plurality of image layers may be used in the respective model 13-15, at least on the input side.

In act S4, a partial scattered radiation image 6 is determined as the physical scatter process by the partial algorithm 26 for a Rayleigh scattering, where the partial attenuation images 22, 23 for the Rayleigh scattering and the Compton scattering and in addition the X-ray image 2 are processed for this purpose.

In act S5, a partial scattered radiation image 7 is determined by the partial algorithm 27 as the physical scatter process for a Compton scattering. The partial attenuation images 23, 24 for the Compton scattering and the photoelectric absorption and in addition the X-ray image 2 are processed for this purpose.

In act S6, a partial scattered radiation image 8 is determined by the partial algorithm 28 as the physical scatter process for a multiple scattering, where all of the partial attenuation images 22-24 and in addition the X-ray image 2 are processed in this case.

In act S7, the partial scattered radiation images 6-8 are added to the estimation 1 for the two-dimensional scattered radiation distribution in the X-ray image 2 (e.g., by an addition of their image values for the respective pixel).

In act S8, optionally, a correction of the X-ray image 2 may then be carried out. In the simplest case, the correction may be performed by subtracting the estimation 1 from the X-ray image 2. However, it is also possible, for example, to carry out an iterative correction (e.g., by iterative multiplication). In principle, in this process, an optimization may be performed within which the image values of the estimation 1 are minimized by variation of the X-ray image 2. If, for example, differentiable models 12-15 trained by machine learning are used in order to implement the estimation algorithm 4, such an optimization may be accomplished, for example, using a gradient descent method.

FIG. 3 shows a flowchart of a possible method for training the trained models 12-15 used in FIG. 2. In act S9, a plurality of training datasets 16 are provided initially for this purpose.

In the example, the respective training dataset 16 includes a two-dimensional X-ray image 2 as source data 17. The respective X-ray image 2 may be provided, for example, by a simulation of a respective imaging procedure (e.g., by solving the Boltzmann transport equation or using a Monte Carlo method).

The respective training dataset further includes, as the respective desired result 19-22, a respective partial scattered radiation image 6-8 that is to be provided by the estimation algorithm 4 during a processing of the source data 17 of the respective training dataset 16. If the X-ray image 2 of the respective training dataset 16 is generated by simulation, the partial scattered radiation images 6-8 for the different scatter processes already explained above may likewise be determined as well in the course of the simulation. Approaches for determining suitable desired results 19-22 for the training datasets 16 in the event that the X-ray image 2 of the respective training dataset 16 is a true X-ray image acquisition have already been discussed in the general part and will not be repeated here.

Acts S10 to S14 are performed for the respective training dataset 16 in order to determine a plurality of partial scattered radiation images 6-8 from the respective X-ray image 2 as an actual result for the current training status of the models 12-15 that are to be trained. The partial scattered radiation images 6-8 may essentially be determined in the course of the training in the same way as has already been explained in relation to FIG. 1. Apart from the training status of the models 12-14, acts S10 to S14 therefore correspond to acts S2 to S6 in FIG. 2.

In act S10, the X-ray image 2 is converted into an intermediate image 5 in order to provide input data 18 that is processed in act S11 by the model 12 to be trained so as to produce the plurality of partial attenuation images 22-24. In acts S12 to S14, a respective image of the partial attenuation images 22-24 is then processed by a respective model of the models 13-15 to be trained so as to produce a respective partial scattered radiation image 6-8.

In act S15, a measure 42 for the deviation of the partial attenuation images 22-24 from the desired results 19 of the respective training dataset 16 may then be determined.

In a simplified variant of the method illustrated in FIG. 3, the measure 42 may be minimized directly as a cost function in order to train the models 12-15. This could be achieved, for example, by a backpropagation in order to realize a supervised learning based on the training datasets 16. If differentiable models 12-15 are used, as is common practice in the field of machine learning, a gradient descent method may be used, for example, in order to minimize the measure 42.

However, in order to take physical characteristics of the interaction and scatter processes that determine the actual scattered radiation distribution into account explicitly in the training, in the example embodiment according to FIG. 3, in acts S16 to S18, further terms of a cost function 30 that may be, for example, a weighted sum of the measure 42 determined in step S15 and of the further terms, are determined in order then, in act S19, to optimize this in the course of the training in the manner already explained in relation to act S15.

In act S16, as such an additional term of the cost function 30, an attenuation deviation 31 is determined for the respective training dataset 16. The attenuation deviation 31 specifies a measure for the deviation of the image values of the pixels of the X-ray attenuation image 29 determined in the example as an intermediate image 5 from a respective comparative value, where the comparative value corresponds to the sum of the image values of all of the partial attenuation images 22-24 for the respective pixel. Since, in the example, a Rayleigh scattering, a Compton scattering, and a photoelectric absorption, and consequently all the interaction processes characterizing the absorption during the X-ray imaging, are taken into account as interaction processes for which a respective partial attenuation image 22-24 is determined. The attenuation deviation should be at least approximately zero if the partial attenuation images 22-24 are determined correctly. Accordingly, the attenuation deviation 31 may be used, for example, as a weighted summand of a cost function 30 that is to be minimized, or the side condition, that the attenuation deviation 31 is zero, may be incorporated into the cost function 30 with the aid of a Lagrange multiplier.

In act S17, for further physical motivation of the training, partial attenuation estimations 32-34 that are based on a classification of the matter 25 irradiated by the X-ray radiation based on the X-ray attenuation image 29 determined in act S10 are determined. In this case, each of the partial attenuation estimations 32-34 describes a local attenuation of the X-ray radiation assumed based on the classification by a respective process of the interaction processes, for which the partial attenuation images 22-24 are also determined.

In act S18, a measure 43 is then determined for the deviation of the respective partial attenuation estimation 32-34 from the respective partial attenuation image 22-24 for the same physical interaction. By taking account of this measure 43 in the cost function 30, the trained model 12 may be trained such that excessively strong deviations of the partial attenuation images 22-24 from the partial attenuation estimations 32-34 are suppressed.

In a development (not shown) of the training method shown in FIG. 3, the respective training dataset 16 may additionally include target images for the partial attenuation images 22-24 and, as part of the cost function 30, a respective measure for the deviation of the respective partial attenuation image 22-24 from a respectively assigned image of the target images may be taken into account. The target images may be determined, for example, in the course of that simulation that is also used in order to determine the desired results 19-21 of the respective training dataset 16.

By this, for example, an end-to-end-training of the entire estimation algorithm 4 may be combined with a training of the first subalgorithm 10 or of the model 12 supervised directly by the target images.

In principle, the consideration of target images specified by the respective training dataset 16 may replace the consideration of the partial attenuation estimations 32-34 determined in act S17 since the target images typically predict the interactions of the X-ray radiation with the matter contributing to the absorption considerably more accurately than this is possible solely based on a classification of image regions in the X-ray image 2 or in the X-ray attenuation image 29. However, it has been recognized that additionally taking account of the attenuation estimation 32-34 also when such target images are used may contribute toward avoiding an overfitting of the trained model 12 and consequently may serve as a regularization.

An example of a possible structure of a trained model 12-15 is shown in FIG. 5. For the sake of simplicity, the structure is illustrated for the use of precisely one image layer for the input data and output data.

The input data for the trained model consists of a two-dimensional medical image having 512×512 pixels, where each pixel contains an image value. The trained model consists of convolutional layers (denoted by solid horizontal arrows), pooling layers (denoted by solid downward-pointing arrows), and upsampling layers (denoted by solid upward-pointing arrows). The number of the respective nodes is indicated in the boxes. Within the U-Net structure, the input data is initially downsampled (e.g., reduction in the size of the images and increase in the number of channels). Subsequently, the input data is upsampled (e.g., enlargement of the images and reduction in the number of channels) in order to generate a transformed image.

All of the convolutional layers L.1, L.2, L.4, L.5, L.7, L.8, L.10, L.11, L.13, L.14, L.16, L.17, L.19, L.20 except for the last convolutional layer L.21 use 3×3 convolutional kernels with a padding of 1, a ReLU activation function, and a number of filters or convolutional kernels corresponding to the number of channels of the respective layers, as indicated in FIG. 5. The last convolutional layer L.22 uses a 1×1 convolutional kernel without padding and the ReLU activation function.

The pooling layers L.3, L.6, L.9 are max pooling layers that replace four neighboring nodes with just one node, where the value is the maximum of the values of the four neighboring nodes. The upsampling layers L.12, L.15, L.18 are transposed convolutional layers having 3×3 kernels and stride 2 that effectively quadruple the number of nodes. The dashed horizontal arrows correspond to concatenation operations in which the output of a convolutional layer L.2, L.5, L.8 of the downward sampling branch of the U-Net structure is used as additional input for a convolutional layer L.13, L.16, L.19 of the upward sampling branch of the U-Net structure. This additional input data is treated as additional channels in the input node layer for the convolutional layer L.13, L.16, L.19 of the upsampling branch.

In the foregoing description, independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for determining an estimation of a two-dimensional scattered radiation distribution in a two-dimensional X-ray image, the computer-implemented method being based on an imaging by an X-ray detector and comprising:
receiving the two-dimensional X-ray image;
applying an estimation algorithm to the two-dimensional X-ray image or to a two-dimensional intermediate image determined from the two-dimensional X-ray image, wherein the estimation algorithm determines a respective two-dimensional partial scattered radiation image for a plurality of physical scatter processes, such that image values of pixels of the respective partial scattered radiation image in each case describe an estimated value for a respective scattered radiation dose that was radiated in onto a respective detector region of the X-ray detector assigned to the respective pixel during acquisition of the X-ray image by the respective physical scatter process; and
determining the estimation of the scattered radiation distribution based on the plurality of two-dimensional partial scattered radiation images.

2. The computer-implemented method of claim 1, wherein the estimation algorithm or at least a subalgorithm of the estimation algorithm is or comprises a model trained by machine learning.

3. The computer-implemented method of claim 2, wherein the model trained by machine learning is based on a supervised training using training datasets, each of which comprises source data and a plurality of desired results,
wherein the source data serves directly or after a preprocessing as input data for the estimation algorithm,
wherein the respective desired result is to be provided by the estimation algorithm as the respective partial scattered radiation image during a processing of the respective input data, and
wherein at least the desired results are based on a simulation of the X-ray imaging.

4. The computer-implemented method of claim 1, wherein separate partial scattered radiation images for at least two or all of the physical scatter processes from the group comprising Rayleigh scattering, Compton scattering, and multiple scattering are determined by the estimation algorithm.

5. The computer-implemented method of claim 1, wherein the estimation algorithm comprises a first subalgorithm that processes the X-ray image or the intermediate image as input data and determines a respective partial attenuation image for a plurality of physical interaction processes of the X-ray radiation with irradiated matter as output data,
wherein image values of the respective partial attenuation image in each case describe an estimation of the local attenuation of the X-ray radiation by the respective physical interaction process, and
wherein a second subalgorithm of the estimation algorithm determines the partial scattered radiation images as a function of the partial attenuation images.

6. The computer-implemented method of claim 5, wherein partial attenuation images for at least two or all of the physical interaction processes are determined from the group comprising Rayleigh scattering, Compton scattering, and photoelectric absorption.

7. The computer-implemented method of claim 6, wherein the second subalgorithm comprises a plurality of partial algorithms,
wherein a respective partial algorithm of the partial algorithms:
determines a partial scattered radiation image for a Rayleigh scattering as the physical scatter process and processes the partial attenuation images for the Rayleigh scattering and the Compton scattering;
determines a partial scattered radiation image for a Compton scattering as the physical scatter process and processes the partial attenuation images for the Compton scattering and the photoelectric absorption;
determines a partial scattered radiation image for a multiple scattering as the physical scatter process and processes the partial attenuation images for the Compton scattering, the Rayleigh scattering, and the photoelectric absorption; or
any combination thereof.

8. The computer-implemented method of claim 5, wherein the first subalgorithm, the second subalgorithm, or the first subalgorithm and the second subalgorithm are in each case a model trained by machine learning, or comprise at least one model trained by machine learning,
wherein the respective trained model is based on a supervised training using training datasets, each of which comprises source data and a plurality of desired results,
wherein the source data serves directly or after a preprocessing as input data for the estimation algorithm and consequently for the first subalgorithm, and
wherein the respective desired result is to be provided as the respective partial scattered radiation image during a processing of the respective input data by the estimation algorithm and consequently by the second subalgorithm.

9. The computer-implemented method of claim 8, wherein the training comprises an optimization of a cost function,
wherein the cost function is dependent on image values of the pixels of the partial attenuation images, the optimization is performed subject to a side condition evaluating the image values of the partial attenuation images, or a combination thereof.

10. The computer-implemented method of claim 9, wherein the fulfillment of the side condition, the cost function, or the side condition and the cost function is:
dependent on an attenuation deviation for the respective training dataset that specifies a measure for the deviation of the image values of the pixels of an X-ray attenuation image specified by the respective source data or determined from the source data from a respective comparative value, wherein the comparative value corresponds to a sum of the image values of all the partial attenuation images for the respective pixel;
dependent on at least one partial attenuation estimation determined for the respective training dataset, wherein the at least one partial attenuation estimation is based on a classification of the matter irradiated by the X-ray radiation exclusively based on the X-ray image or X-ray attenuation image specified by the source data of the respective training dataset, wherein the respective partial attenuation estimation describes an estimated attenuation of the X-ray radiation by a respective process of the interaction processes in the respective pixel of the X-ray image or X-ray attenuation image; or
a combination thereof.

11. A computer-implemented method for training at least one trained model by which an estimation algorithm or at least one respective subalgorithm or partial algorithm of the estimation algorithm is implemented, using machine learning, the computer-implemented method comprising:
receiving training datasets, each of which comprises source data and a plurality of desired results, wherein the source data in each case is or comprises a two-dimensional X-ray image or a two-dimensional intermediate image that is based on an X-ray imaging procedure or a simulation of an X-ray imaging procedure, and wherein the respective desired result specifies a respective partial scattered radiation image that is to be provided by the estimation algorithm during a processing of the source data of the respective training dataset;
training the at least one trained model using a supervised learning based on the training datasets; and
providing the at least one trained model.

12. A processing device for determination of an estimation of a two-dimensional scattered radiation distribution in a two-dimensional X-ray image, the determination being based on an imaging by an X-ray detector, the processing device comprising:
a processor configured to:
receive the two-dimensional X-ray image;
apply an estimation algorithm to the two-dimensional X-ray image or to a two-dimensional intermediate image determined from the two-dimensional X-ray image, wherein the estimation algorithm determines a respective two-dimensional partial scattered radiation image for a plurality of physical scatter processes, such that image values of pixels of the respective partial scattered radiation image in each case describe an estimated value for a respective scattered radiation dose that was radiated in onto a respective detector region of the X-ray detector assigned to the respective pixel during acquisition of the X-ray image by the respective physical scatter process; and
determine the estimation of the scattered radiation distribution based on the plurality of two-dimensional partial scattered radiation images.

13. An X-ray device comprising:
an X-ray source;
an imaging X-ray detector; and
a processing device for determination of an estimation of a two-dimensional scattered radiation distribution in a two-dimensional X-ray image, the determination being based on an imaging by the imaging X-ray detector, the processing device comprising:
a processor configured to:
receive the two-dimensional X-ray image;
apply an estimation algorithm to the two-dimensional X-ray image or to a two-dimensional intermediate image determined from the two-dimensional X-ray image, wherein the estimation algorithm determines a respective two-dimensional partial scattered radiation image for a plurality of physical scatter processes, such that image values of pixels of the respective partial scattered radiation image in each case describe an estimated value for a respective scattered radiation dose that was radiated in onto a respective detector region of the imaging X-ray detector assigned to the respective pixel during acquisition of the X-ray image by the respective physical scatter process; and
determine the estimation of the scattered radiation distribution based on the plurality of two-dimensional partial scattered radiation images.

14. In a non-transitory computer-readable storage medium that stores instructions executable by one or more processors to determine an estimation of a two-dimensional scattered radiation distribution in a two-dimensional X-ray image, the determination being based on an imaging by an X-ray detector, the instructions comprising:
receiving the two-dimensional X-ray image;
applying an estimation algorithm to the two-dimensional X-ray image or to a two-dimensional intermediate image determined from the two-dimensional X-ray image, wherein the estimation algorithm determines a respective two-dimensional partial scattered radiation image for a plurality of physical scatter processes, such that image values of pixels of the respective partial scattered radiation image in each case describe an estimated value for a respective scattered radiation dose that was radiated in onto a respective detector region of the X-ray detector assigned to the respective pixel during acquisition of the X-ray image by the respective physical scatter process; and
determining the estimation of the scattered radiation distribution based on the plurality of two-dimensional partial scattered radiation images.

15. The non-transitory computer-readable storage medium of claim 14, wherein the estimation algorithm or at least a subalgorithm of the estimation algorithm is or comprises a model trained by machine learning.

16. The non-transitory computer-readable storage medium of claim 15, wherein the model trained by machine learning is based on a supervised training using training datasets, each of which comprises source data and a plurality of desired results,
wherein the source data serves directly or after a preprocessing as input data for the estimation algorithm,
wherein the respective desired result is to be provided by the estimation algorithm as the respective partial scattered radiation image during a processing of the respective input data, and
wherein at least the desired results are based on a simulation of the X-ray imaging.

17. The non-transitory computer-readable storage medium of claim 14, wherein separate partial scattered radiation images for at least two or all of the physical scatter processes from the group comprising Rayleigh scattering, Compton scattering, and multiple scattering are determined by the estimation algorithm.

18. The non-transitory computer-readable storage medium of claim 14, wherein the estimation algorithm comprises a first subalgorithm that processes the X-ray image or the intermediate image as input data and determines a respective partial attenuation image for a plurality of physical interaction processes of the X-ray radiation with irradiated matter as output data,
wherein image values of the respective partial attenuation image in each case describe an estimation of the local attenuation of the X-ray radiation by the respective physical interaction process, and
wherein a second subalgorithm of the estimation algorithm determines the partial scattered radiation images as a function of the partial attenuation images.

19. The non-transitory computer-readable storage medium of claim 18, wherein partial attenuation images for at least two or all of the physical interaction processes are determined from the group comprising Rayleigh scattering, Compton scattering, and photoelectric absorption.
